(19) United States
(12) Patent Application Publication
(10) Pub. No.: US 2014/0294974 A1
(43) Pub. Date: Oct. 2, 2014

(54) COSMETIC COMPOSITION

(71) Applicant: Conopco, inc, d/b/a UNILEVER, Englewood Cliffs, NJ (US)

(72) Inventors: Ezat Khoshdel, Merseyside (GB); Qiqing Zhang, Shanghai (CN)

(73) Assignee: CONOPCO, INC. D/B/A UNILEVER, Englewood Cliffs, NJ (US)

(21) Appl. No.: 14/354,603

(22) PCT Filed: Oct. 16, 2012
(86) PCT No.: PCT/EP2012/070485

§ 371 Date: Apr. 28, 2014

(30) Foreign Application Priority Data
Nov. 4, 2011 (CN) PCT/CN2011/081810

(51) Int. Cl. A61K 47/02; A61Q 5/00; A61Q 5/02; A61K 31/465

(52) U.S. Cl. CPC A61K 47/02; A61K 31/465; A61Q 5/006; A61Q 5/02
USPC 424/489; 514/355

(57) ABSTRACT

The invention relates to a cosmetic composition comprising an emulsion comprising hydrophobically modified particles, amino functionalized silicone and water; the cosmetic composition further comprising a moisturizing agent and at least one component selected from the group consisting of vitamin, anti-dandruff agent and surfactant, wherein the composition comprises at least a vitamin or anti-dandruff agent. The invention further relates to a method of treating a skin condition comprising the step of topically applying to the skin the cosmetic composition of the invention.

### FIELD OF THE INVENTION

The present invention relates to cosmetic compositions, particularly cosmetic compositions suitable for improving skin health.

### BACKGROUND OF THE INVENTION

Many consumers are concerned with the characteristics of their skin. Some consumers may feel they have dry skin, dull looking skin or other imperfections like for examples wrinkles and fine lines.

To meet the needs of consumers products have been developed that address skin health and improve skin characteristics. Such skin care compositions usually contain at least a moisturizing agent like for example glycerin. In addition to moisturizing agents other ingredients are used like for example vitamins and anti-dandruff agents that address specific skin characteristics.

Consumers prefer products that are easy to use (like for example easy to apply to skin) and/or deliver excellent sensory benefits after topical application to skin (like for example non-stickiness or a silky sensation).

Moisturizing agents may leave a sticky feeling after application to skin.

There is thus a need for cosmetic compositions with good sensory properties like for example non-stickiness.

The inventors have developed cosmetic compositions that do not suffer from the aforementioned drawbacks.

### SUMMARY OF THE INVENTION

Accordingly the present invention relates to a cosmetic composition comprising an emulsion comprising
a. hydrophobically modified particles selected from the group consisting of silica, metal oxide and mixtures thereof;
b. amino functionalized silicone; and
c. water;
   the cosmetic composition further comprising a
d. moisturizing agent; and
e. at least one component selected from the group consisting of vitamin, anti-dandruff agent and surfactant;
   wherein the composition comprises at least a vitamin or anti-dandruff agent.

The invention further relates to a method of treating a skin condition comprising the step of topically applying the cosmetic composition of the invention.

### DETAILED DESCRIPTION OF THE INVENTION

Weight percentage (wt %) is based on total weight of composition unless otherwise stated.

The cosmetic composition of the invention is an emulsion and can be water in oil or oil in water emulsions. Preferred emulsions are oil in water emulsions. Preferably the level of water is from 10 to 90 wt %, more preferably 20 to 85 wt %, even more preferably 30 to 80 wt % and still even more preferably 40 to 70 wt %.

Hydrophobically Modified Particles

The compositions of the invention comprise hydrophobically modified particles of silica, metal oxide and mixtures thereof. Preferably the hydrophobically modified particles comprise silica or titanium oxide, more preferably hydrophobically modified silica (silicone dioxide).

It is particularly preferred if the hydrophobically modified silica comprises the hydrophobic group of formula:

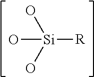

in which R is a C₄ to C₁₈ alkyl group.

Particularly preferred is the hydrophobically modified silicone dioxide comprising:

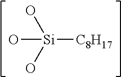

Preferred silicas are described in U.S. Pat. No. 7,282,236 and made commercially available from suppliers like Evonik Degussa GmbH under the names Aerosil R812, R202 and R805. Silica having a C4 to C15 alkyl group attached to the silane are preferred. Particularly preferred is octylsilane comprising the group represented by the formula above (sold under the name Aerosil R805).

The hydrophobically modified silica preferably has a primary particle size (in its dry state) from 1 nm to 100 nm, more preferably from 5 nm to 70 nm.

Composition of the invention preferably comprise from 0.05 to 15 wt % of the total composition of hydrophobically modified silica, more preferably from 0.1 to 10 wt %, and most preferably from 0.2 to 5 wt %.

Amino Functionalized Silicone

Compositions according to the invention comprise an amino functional silicone. By “amino functionalized silicone” is meant a silicone containing at least one primary, secondary or tertiary amine group, or a quaternary ammonium group.

The primary, secondary, tertiary and/or quaternary amine groups may either form part of the main polymer chain or more preferably be carried by a side or pendant group carried by the polymeric backbone.

Suitable amino functionalized silicone polymers for use with the invention are described in U.S. Pat. No. 4,185,087.

Amino functionalized silicones suitable for use in the invention will typically have a mole % amine functionality in the range of from about 0.1 to about 8.0 mole %, preferably from about 0.1 to about 5.0 mole %, most preferably from about 0.1 to about 2.0 mole %. In general the amine concentration should not exceed about 8.0 mole %.

In a preferred embodiment, the functionalized polymers are of the amodimethicone having the general formula:

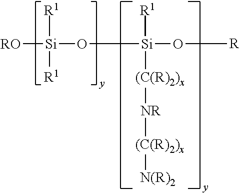

where each R is independently H, or a C₁₋₄ alkyl, preferably H;

each R¹ is independently OR or a C₁₋₄ alkyl; and

each x is independently an integer from 1 to 4 and each y is greater than zero and independently an integer to yield a polymer having a molecular weight from 500 to 1 million, and preferably from 750 to 25,000, and most preferably from 1,000 to 15,000.

Particularly preferred are silicones comprising an amino glycol copolymer. Especially preferred is Bis (C13-C15 alkoxy) PG amodimethicone such as DC 8500 by Dow Corning.

It is also within the scope of this invention for the functionalized polymer to comprise trimethylsilylamodimethicone.

Preferably the level of amino functionalized silicone polymers make up from 0.05 to 25 wt % of the total composition, more preferably from 0.05 to 15 wt %, even more preferably from 0.1 to 10%, and still even more preferably from 0.2 to 5 wt %.

Preferably the weight ratio of hydrophobically modified particle to amino functionalized silicone is from 3:1 to 1:50, more preferably from 1:1 to 1:10.

Moisturizing Agent

Compositions of the invention comprise a moisturizing agent. Preferably the moisturizing agent is selected from the group consisting of polyhydric alcohols, substituted ureas and mixtures thereof.

Typical polyhydric alcohols include glycerin (i.e. glycerol), propylene glycol, dipropylene glycol, polypropylene glycol, polyethylene glycol, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof.

Typical substituted ureas are hydroxymethyl urea, hydroxyethyl urea, hydroxypropyl urea; bis(hydroxymethyl)urea; bis(hydroxyethyl)urea; bis(hydroxypropyl)urea; N,N′-dihydroxymethyl urea; N,N′-di-hydroxyethyl urea; N,N′-di-hydroxypropyl urea; N,N,N′-tri-hydroxyethyl urea; tetra(hydroxymethyl)urea; tetra(hydroxyethyl)urea; tetra(hydroxypropyl urea; N-methyl, N′-hydroxyethyl urea; N-ethyl-N′-hydroxyethyl urea; N-hydroxypropyl-N′-hydroxyethyl urea and N,N′dimethyl-N-hydroxyethyl urea or mixtures thereof. Where the term hydroxypropyl appears, the meaning is generic for either 3-hydroxy-n-propyl, 2-hydroxy-n-propyl, 3-hydroxy-i-propyl or 2-hydroxy-i-propyl radicals. Most preferred is hydroxyethyl urea. The latter is available as a 50% aqueous liquid from the National Starch & Chemical Division of ICI under the trademark Hydrovance.

Preferably the moisturizing agent comprises at least a polyhydric alcohol. Preferably the polyhydric alcohol is glycerin.

Preferably the level of moisturizing agent is from 0.01 to 25 wt %, preferably 0.2 to 20 wt %, more preferably 1 to 15 wt %, and even more preferably 2 to 10 wt %.

Vitamin, Anti-Dandruff Agent, Surfactant

Compositions of the invention comprise at least one component selected from the group consisting of vitamin, anti-dandruff agent and surfactant, wherein the compositions of the invention comprise at least a vitamin or anti-dandruff agent.

Illustrative vitamins are Vitamin A (retinol), Vitamin B2, Vitamin B6, Vitamin C, Vitamin E, Folic Acid and Biotin. Derivatives of the vitamins may also be employed. For instance, Vitamin C derivatives include ascorbyl tetraisopalmitate, magnesium ascorbyl phosphate and ascorbyl glycoside. Derivatives of Vitamin E include tocopheryl acetate, tocopheryl palmitate and tocopheryl linoleate. DL-panthenol and derivatives may also be employed.

Preferably the vitamin is selected from the group consisting of A, B, C, D, E, derivatives thereof and combinations thereof.

Preferably the level of vitamin is from 0.001 to 10 wt %, preferably 0.01 to 8 wt %, more preferably 0.1 to 6 wt %, and even more preferably 1 to 3 wt %.

Antidandruff agents are compounds that are active against dandruff and are typically antimicrobial agents and preferably antifungal agents.

Antifungal agents typically display a minimum inhibitory concentration of about 50 mg/ml or less against Malassezia spp.

Suitable antidandruff agents include compounds selected from ketoconazole, climbazole, octopirox, metal pyrithione salts, and mixtures thereof.

The preferred azole based antifungal agents are ketoconazole and climbazole. Preferred metal pyrithione salts are zinc, copper, silver and zirconium pyrithione. The most preferred is zinc pyrithione.

Preferably, the antidandruff active is present at from 0.01 to 5% wt. of the composition, more preferably from 0.1 to 2.5% wt. of the composition.

Surfactants may also be present in cosmetic compositions of the present invention. Total concentration of the surfactant will range from 0 to 40 wt %, and preferably, from 0.01 to 20 wt %, optimally from 0.01 to 5 wt % of the composition. The surfactant may be selected from the group consisting of anionic, nonionic, cationic and amphoteric actives. Particularly preferred nonionic surfactants are those with a C10-C20 fatty alcohol or acid hydrophobe condensed with from 2 to 100 moles of ethylene oxide or propylene oxide per mole of hydrophobe; C2-C10 alkyl phenols condensed with from 2 to 20 moles of alkylene oxide; mono- and di-fatty acid esters of ethylene glycol; fatty acid monoglyceride; sorbitan, mono- and di-C8-C20 fatty acids; block copolymers (ethylene oxide/propylene oxide); and polyoxyethylene sorbitan as well as combinations thereof. Alkyl polyglycosides and saccharide fatty amides (e.g. methyl gluconamides) are also suitable nonionic surfactants.

Preferred anionic surfactants include soap, alkyl ether sulfate and sulfonates, alkyl sulfates and sulfonates, alkylbenzene sulfonates, alkyl and dialkyl sulfosuccinates, C8-C20 acyl isothionates, acyl glutamates, C8-C20 alkyl ether phosphates and combinations thereof.

Viscosity Modifying Agent

Compositions of the invention preferably also comprise a viscosity modifying agent selected from the group consisting of non-amino functionalized silicone, fatty acids, esters of fatty esters, hydrocarbons, fatty alcohols and mixtures thereof.

Suitable non-amino functionalized silicones include and preferably are polydiorganosiloxanes, in particular polydimethylsiloxanes which have the CTFA designation dimethicone. Also suitable for use in compositions of the invention are polydimethyl siloxanes having hydroxyl end groups, which have the CTFA designation dimethiconol. Also suitable for use in compositions of the invention are silicone gums having a slight degree of cross-linking, as are described for example in WO 96/31188. Suitable non-amino functionalized silicones are volatile silicone oils like for example cyclopentasiloxane (e.g. DC 245 Fluid from Dow Corning); non-volatile polydimethylsiloxane polymers like the DC 200 series of oils from Dow Corning with viscosity from 0.65 cst to 600,000 cst; silicone elastomer blends like DC 9041(dimethicone and dimethicone crosspolymer from Dow Corning), DC 9045 and DC 9040 (cyclopentasiloxane and dimethicone crosspolymer from Dow Corning) DC 9546 (cyclopentasiloxane and dimethicone crosspolymer and dimethicone/vinyldimethicone crosspolymer and dimethiconol, from Dow Corning), DC 9506 (dimethicone/vinyldimethicone crosspolymer from Dow Corning); silicone elastomer suspensions like DC 9509 (dimethicone/vinyldimethicone crosspolymer and C12-14 Pareth-12 from Dow Corning).

Suitable fatty acids are fatty acid from 10 to 30 carbon atoms. Illustrative examples of such fatty acids include pelargonic, lauric, myristic, palmitic, stearic, isostearic, oleic, linoleic, arachidic, behenic or erucic acid, and mixtures thereof.

Esters of fatty acids include and preferably are alkyl ester of saturated fatty acids having 10-24 carbon atoms; ether-esters such as fatty acid esters of ethoxylated saturated fatty alcohols; polyhydric alcohol esters, ethylene glycol mono- and di-fatty acid esters, diethylene glycol mono- and di-fatty acid esters and so on; wax esters such as beeswax, spermaceti wax and tribehenin wax; sugar ester of fatty acids such as sucrose polybehenate and sucrose polycottonseedate; natural ester emollients based on mono- di-tri-glycerides.

Hydrocarbons include and preferably are petrolatum, mineral oil, C11-C13 isoparaffins, polytubenes, isohexadecane available commercially as permethyl 101 A from presperse Inc.

Fatty alcohols are aliphatic alcohols consisting of a chain of at least 8 carbon atoms. Preferably the fatty alcohol has a chain of 10 to 30 carbon atoms.

Preferably the viscosity modifying agent comprises at least a non-amino functionalized silicone and more preferably the viscosity modifying agent is selected from the group consisting of polydimethylsiloxane, pentasiloxane, dimethicone, mineral oil and mixtures thereof.

Preferred viscosity modifying agents are Dimethicone DC 200 series of oils from Dow Corning with viscosities from 0.65 cst to 6000 cst, mineral oil 70 SUS.

Preferably the level of viscosity modifying agent is from 0.05 to 60 wt %, preferably 1 to 40 wt % and more preferably 5 to 30 wt %.

Preferably the weight ratio of viscosity modifying agent to amino functionalized silicone is from 10:1 to 1:50 and preferably from 3:1 to 1:20.

Further Ingredients

In addition to water, organic solvents may be optionally included to act as carriers or to assist carriers within the compositions of the present invention. Illustrative and non-limiting examples of the types of organic solvents suitable for use in the present invention include alkanols like ethyl and isopropyl alcohol, mixtures thereof or the like.

Thickeners may also be utilized as part of the cosmetic compositions of the invention. Typical thickeners include cross-linked acrylates (e.g. Carbopol 982), hydrophobically-modified acrylates (e.g. Carbopol 1382), cellulosic derivatives and natural gums.

Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Amounts of the thickener may range from 0.0 to 5 wt %, usually from 0.001 to 1 wt %, optimally from 0.01 to 0.5 wt %.

Perfumes may be used in the cosmetic composition of this invention. Illustrative non-limiting examples of the types of perfumes that may be used include those comprising terpenes and terpene derivatives like those described in Bauer, K., et al., Common Fragrance and Flavor Materials, VCH Publishers (1990).

Illustrative yet non-limiting examples of the types of fragrances that may be used in this invention include myrcene, dihydromyrenol, citral, tagetone, cis-geranic acid, citronellic acid, or cis-geranic acid nitrile, mixtures thereof or the like.

Preferably, the amount of fragrance employed in the cosmetic composition of this invention is in the range from 0 to 10 wt %, more preferably, 0.00001 to 5 wt %, most preferably, 0.0001 to 2 wt %.

Sunscreens include those materials commonly employed to block ultraviolet light. Illustrative compounds are the derivatives of PABA, cinnamate and salicylate. For example, avobenzophenone (Parsol 1789®) octyl methoxycinnamate and 2-hydroxy-4-methoxyl benzophenone (also known as oxybenzone) can be used. Octyl methoxycinnamate and 2-hydroxy-4-methoxy benzophenone are commercially available under the trademarks, Parsol MCX and Benzophenone-e, respectively. The exact amount of sunscreen employed in the compositions can vary depending upon the degree of protection desired from the sun's UV radiation.

Many cosmetic compositions, especially those containing water, should be protected against the growth of potentially harmful microorganisms. Anti-microbial compounds, such as triclosan, and preservatives are, therefore, typically necessary. Suitable preservatives include alkyl esters of p-hydroxybenzoic acid, hydantoin derivatives, propionate salts, and a variety of quaternary ammonium compounds. Particularly preferred preservatives of this invention are methyl paraben, propyl paraben, phenoxyethanol and benzyl alcohol. Preservatives will usually be employed in amounts ranging from 0.1 to 2 wt % of the composition.

Cosmetic Compositions

Cosmetic composition, as used herein, is meant to include a composition for topical application to skin of mammals, especially humans. Compositions of the invention are typically ‘leave-on’ compositions, and is meant to include conditioners or tonics, and general topical compositions that in some fashion and at the very least relate to and address skin health. Skin health may include skin moisturizing, decreasing the effect of sebum on the skin and skin wrinkle reducing.

Cosmetic compositions of the invention can be in the form of a liquid, lotion, cream, serum or gel. The composition of this invention is one that at the very least addresses the health of skin when skin is meant to include skin on the face, neck, chest, back, arms, hands, legs and preferably scalp.

The viscosity of the final cosmetic composition at 25 degrees Celsius preferably is from 10³ Pa·s to 10⁵ Pa·s, and more preferably 5*10⁴ Pa·s to 5*10⁵ Pa·s.

Often, the cosmetic composition of the present invention has a melting point from about 30 to 45 degrees Celsius. In an especially preferred embodiment, the cosmetic composition of the present invention has a pH from 4.5 to about 8 at 25 degrees Celsius. More preferably from 5 to 7.

Method of Treating a Skin Condition

The invention also concerns a method of treating a skin condition comprising the step of topically applying to the skin the cosmetic composition of the invention as described.

Preferably the composition of the invention is used as a ‘leave-on’ composition.

Preferably the method concerns the cosmetic non-medical treatment of a skin condition and preferably the method addresses dry skin.

The cosmetic composition of the invention is especially suitable to deliver moisturizing agents without leaving a ‘sticky-feeling’ to the skin the cosmetic composition of the invention is applied on. Even when applied to the scalp, the composition of the invention does not interfere with the sensory feeling of hair on the scalp (if present). Therefore the method preferably addresses scalp health.

The invention is now illustrated by the following non-limiting examples.

### EXAMPLES

Primary Particle Size

Primary particle sizes can be derived from Transmission Electron Microscopy according to the method described by S. Gu et al in Journal of Colloid and Interface Science 289 (2005) 419-426.

Viscosity Measurement

Viscosity is a measure of the resistance of fluid to an applied stress. A MCR 501 rheometer (from Anton Paar Instrument Ltd) is used to measure the viscosities. The rheometer is connected to a compressed air source to maintain a pressure of 5 bar, to ensure a continuous rotation rate. A parallel plate was used having geometry of 25 mm in diameter. An automatic controlled rate mode is used to measure the forces in a shear rate range from 0.001 to 100 s⁻¹ at a temperature of 25 degrees Celsius.

Formulation Process

The examples are made by mixing the silica particles in the silicone oils. The remaining ingredients are added into the oil phase by stirring. Comparative A is made by mixing the ingredients. All samples were made under ambient temperature.

Ingredients Used

Aerosil R805 ex. Evonik Degussa GmbH; DC 8500, DC 245, DC 200 fluid 5cst, all ex. Dow Corning.

### Example 1

TABLE 1
Composition of example 1 (in wt %)

| Trade name | Chemical name | Ex. 1 |
|---|---|---|
| Water | Water | to 100 |
| Carbopol Ultrez 20 | Acrylates/C10-30 Alkyl Acrylate | 0.40 |
| | Crosspolymer | |
| NaOH (25%) | Sodium Hydroxide (25%) | 0.40 |
| Glydant (55%) | 1,3-bis(hydroxymethyl)-5,5-dimethyl | 0.20 |
| EDTA 4Na (39%) | ethylenediamine tetraacetic acid | 0.13 |
| | sodium | |
| Aerosil R805 | Hydrophobic silica | 0.1 |
| DC 8500 | Bis (C13-15Alkoxy) PG | 0.1 |
| | Amodimethicone | |
| DC 200 fluid 5 cst | Polydimethylsiloxane | 0.3 |
| DC 245 | Cyclopentasiloxane | 0.50 |
| Ethanol | Ethyl alcohol | 5.00 |
| Niacinamide Vitamin B3 | Niacinamide | 5.00 |
| Glycerin | Glycerin | 5.00 |

Comparative A

TABLE 2
Composition of comparative A (in wt %)

| Trade name | Chemical name | Comp. A |
|---|---|---|
| Water | Water | to 100 |
| Carbopol Ultrez 20 | Acrylates/C10-30 Alkyl Acrylate | 0.40 |
| | Crosspolymer | |
| NaOH (25%) | Sodium Hydroxide (25%) | 0.40 |
| Glydant (55%) | 1,3-bis(hydroxymethyl)-5,5-dimethyl | 0.20 |
| EDTA 4Na (39%) | ethylenediamine tetraacetic acid | 0.13 |
| | sodium | |
| Rhodasurf L790 (90%) | Sodium C12-15 Pareth Sulfate | 1.11 |
| EumulginL | PPG-1-PEG-9 Lauryl Glycol Ether | 3.00 |
| DC 245 | Cyclopentasiloxane | 0.50 |
| DC 1785 (50%) | Dimethiconol (and) TEA- | 0.072 |
| | Dodecylbenzenesulfonate | |
| Ethanol | Ethyl alcohol | 5.00 |
| Niacinamide Vitamin | Niacinamide | 5.00 |
| B3 | | |
| Glycerin | Glycerin | 5.00 |

Attributes

Example 1 and comparative A were assessed for spreadibility, stickiness and ease of absorbance by applying the composition to scalp skin (32 persons). Each person scored their preference against the three attributes.

TABLE 3
Attributes of example 1 and comparative A

| Attribute | Ex. 1 | Comp. A |
|---|---|---|
| Spreadibility | 24 | 8 |
| Less sticky | 23 | 9 |
| Ease of absorbance | 22 | 10 |

Example 1 (according to the invention) had a better performance on all attributes compared with comparative A (not according to the invention).

### Examples 2 and 3

A leave-on anti-dandruff serum (example 2) and leave-on anti-dandruff conditioner (example 3), both according to the invention, were formulated.

TABLE 4
Composition of examples 2 and 3 (in wt %)

| Trade name | Chemical name | Ex. 2 | Ex. 3 |
|---|---|---|---|
| Water | Water | to 100 | to 100 |
| Carbopol Ultrez 20 | Acrylates/C10-30 Alkyl Acrylate | 0.40 | — |
| | Crosspolymer | | |
| NaOH | Sodium Hydroxide | 0.20 | — |
| TEA | TEA | — | 0.08 |
| Preservatives | preservative | 0.10 | 0.20 |
| Aerosil R805 | Hydrophobic silica | 0.10 | 0.07 |
| DC 8500 | Bis (C13-15Alkoxy) PG | 0.10 | 0.07 |
| | Amodimethicone | | |
| DC 200 fluid 5 cst | Polydimethylsiloxane | 0.30 | 0.20 |
| DC 245 | Cyclopentasiloxane | 0.50 | 0.17 |
| Ethanol | Ethyl alcohol | 5.00 | 2.00 |
| Glycerin | Glycerin | 10.00 | 10.00 |
| Eumulgin L | PPG-1-PEG-9 Lauryl Glycol | 3.00 | — |
| | Ether | | |
| Rhodasurf L790 | Laureth-7 | 1.11 | — |
| BTAC DPG | Behentrimonium Chloride and | — | 0.50 |
| | Dipropylene Glycol | | |
| Hydrenol MY | Cetearyl Alcohol | — | 1.50 |
| Climbazole | Climbazole | 0.30 | — |
| ZPTO(48%) | Zinc Pyrithione | — | 0.42 |
| — | Salicylic Acid | — | 0.20 |

1. A cosmetic composition comprising an emulsion comprising
a. hydrophobically modified particles selected from the group consisting of silica, metal oxide and mixtures thereof;
b. amino functionalized silicone; and
c. water;
the cosmetic composition further comprising
d. a moisturizing agent; and
e. at least one component selected from the group consisting of vitamin, anti-dandruff agent and surfactant;
wherein the composition comprises at least a vitamin or anti-dandruff agent.

2. A composition according to claim 1 wherein the hydrophobically modified particles comprise silica or titanium oxide, more preferably comprise silica.

3. A composition according to claim 1 wherein the hydrophobically modified silica comprise 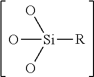
wherein R is a C4 to C18 alkyl group, more preferably a C8H17 alkyl group.

4. A composition according to claim 1 wherein the hydrophobically modified particle has a primary particle size from 1 nm to 100 nm, preferably from 5 nm to 70 nm.

5. A composition according to claim 1 wherein the amino functionalized silicone comprises an amino glycol copolymer.

6. A composition according to claim 1 wherein the level of amino functionalized silicone is from 0.05 to 25 wt %, preferably 0.05 to15 wt %, more preferably 0.1 to 10 wt % and even more preferably 0.2 to 5 wt % of the total composition.

7. A composition according to claim 1 wherein the weight ratio of hydrophobically modified particle to amino functionalized silicone is from 3:1 to 1:50, preferably from 1:1 to 1:10.

8. A composition according to claim 1 wherein the moisturizing agent is selected from the group consisting of polyhydric alcohols, substituted ureas and mixtures thereof.

9. A composition according to claim 1 wherein the moisturizing agent comprises at least a polyhydric alcohol, and preferably the polyhydric alcohol is glycerin.

10. A composition according to claim 1 wherein the level of moisturizing agent is from 0.01 to 25 wt %, preferably 0.2 to 20 wt %, more preferably 1 to 15 wt %, and even more preferably 2 to 10 wt %.

11. A composition according to claim 1 further comprising a viscosity modifying agent selected from the group consisting of non-amino functionalized silicone, fatty acids, esters of fatty esters, hydrocarbons, fatty alcohols and mixtures thereof, and wherein the weight ratio of viscosity modifying agent to amino functionalized silicone preferably is from 10:1 to 1:50 and more preferably from 3:1 to 1:20.

12. A composition according to claim 1 wherein the vitamin is selected from the group consisting of A, B, C, D, E, derivatives thereof and combinations thereof.

13. A composition according to claim 1 wherein the level of vitamin is from 0.001 to 10 wt %, preferably 0.01 to 8 wt %, more preferably 0.1 to 6 wt %, and even more preferably 1 to 3 wt %.

14. A composition according to claim 1 wherein the viscosity of the composition at 25 degrees Celsius is from 10³ Pa·s to 10⁵ Pa·s, and preferably 5*10⁴ Pa·s to 5*10⁵ Pa·s.

15. A method of treating a skin condition comprising the step of topically applying to the skin the cosmetic composition of claim 1.
